(19) United States
(12) Patent Application Publication
(10) Pub. No.: US 2020/0107902 A1
(43) Pub. Date: Apr. 9, 2020

(54) Osteotomy Device

(71) Applicant: Daniel Ezra Walzman, Bergenfield, NJ (US)

(72) Inventor: Daniel Ezra Walzman, Bergenfield, NJ (US)

(21) Appl. No.: 16/151,335

(22) Filed: Oct. 3, 2018

(51) Int. Cl. A61B 90/00; A61B 1/00; A61B 1/015; A61B 1/05; A61B 17/15

(52) U.S. Cl. CPC A61B 90/04; A61B 90/37; A61B 1/00009; A61B 1/00071; A61B 2018/00595; A61B 1/015; A61B 1/05; A61B 17/151; A61B 1/00147; A61B 1/00119

(57) ABSTRACT

The present disclosure teaches a novel medical device for osteotomy, capable of simultaneous aspiration, cauterization, image-acquisition, irrigation and tissue-protection, adapted for ear, nose and throat procedures, but available for other cavity surgeries.

### FIELD OF THE INVENTION

The described invention relates generally to medical devices, systems and methods used for surgery to cut, excise or otherwise shape bones, and more particularly to methods and devices for performing image guided interventional procedures to treat disorders of the paranasal sinuses, ears, nose or throat (ENT).

### BACKGROUND OF THE INVENTION

### Prior Art

Osteotomy devices are numerous. They include devices for cauterizing, irrigating, aspirating, image-collection, and cutting, among others. These devices are generally standalone, and normally can only be used in pairs, occasionally threes. The imaging is of two types: the first involves external sensors with tagged instruments; the second involves image-collection, either prior to the surgery or mounted on an insertion device.

Image-guided surgical techniques and devices were developed for neurosurgery and have now been adapted for use in certain ear, nose and throat surgeries, including sinus surgeries. See, Kingdom T. T., Orlandi R. R., Image-Guided Surgery of the Sinuses: Current Technology and Applications, Otolaryngol. Clin. North Am. 37(2):381-400 (April 2004). Generally, image-guided surgery involves getting images prior to surgery and then using said images to help the surgeon to execute.

While Image guided surgery typically employs electromagnetic sensors/tracking systems, radiofrequency electromagnetic sensors (e.g., electromagnetic coils) which are placed on the surgical instruments and on a localizer frame worn by the patient, they do not have built in irrigation and respiration systems.

The lack of built-in irrigation and aspiration systems is a shortcoming of the prior art. Image-guided surgery systems are used in sinus surgery and other ear, nose and throat procedures. More particularly, this shortcoming results in the requirement that the surgeon insert and remove separate irrigation and respiration systems causing delay in procedures. The presence of said separate systems causes sensors which have been mounted on proximal portions of the instruments (e.g., on the handpiece of the instrument) to be moved away from targeted surgery sites resulting in the reduction of the accuracy of sensor information.

There is a need to perform minimally invasive osteotomies, especially for ear, nose and throat sinus procedures, in a safe and less invasive manner, with smaller instruments, and direct visualization, to facilitate safe performance of these procedures with less need for general anesthesia and its associated risks, and less need for the costs associated with performance of these procedures in an OR (increased ability to perform in office setting, with lower costs). Also allows for protection of surrounding tissue during bone cuts. Currently only select balloon sinu-plasty procedures are performed in this setting, but these are less effective at relieving symptoms, and have much higher rates of recurrent symptomatic sinus blockage/infections.

Additionally, a need exists to protect tissue near surgical target-sites. While some use of insertable shielding is described in the prior art, such shielding is difficult to position, tends to migrate out of position, and can cause irritation to tissue.

Therefore, a need exists for an osteotomy device or system which is faster, more efficient, and easier to use. A need exists to better protect tissue proximal a surgical target during osteotomic procedures. A need exists for an osteotomy device comprising one or more modalities used in non-operating theater settings.

### SUMMARY OF THE INVENTION

The present invention combines numerous osteotomy devices as well as adding a device not described in the prior art. More particularly, the present invention combines a shielding device for tissue proximal to the targeted surgical site, a cutting device, a cauterization device, an aspiration device, an irrigation device, cameras/endoscopes, lighting into an optionally malleable and/or steerable device. This multifunctional device allows the simultaneous application of cutting, irrigating, aspirating, cauterizing, while feeding back live visual information to the user, while minimizing tissue injury.

According to one aspect, the described invention provides irrigation and aspiration systems which are integrated into devices that are useable to perform image-guided procedures as well as a variety of other image guided ear, nose and throat procedures.

Additionally, the present invention provides improvements and modifications to the prior art's image-guided surgery systems to facilitate the performance of image-guided surgery and other image ear, nose and throat procedures with mini al or less iatrogenic trauma to and/or alteration of anatomical structures that are not involved in the disorder being treated.

### BRIEF DESCRIPTION OF THE DRAWINGS

FIG. 1 illustrates an exemplary embodiment of the osteotomy device 1 of the disclosed invention, including nonoptional elements: distal hole 1000, proximal hole 2000, shielding element 11, a side-hole 10, side-camera 30 and end-camera 20, protective or hydrogel coating 40, and an optional square handle 90, optionally detachable, disposed outside the patient's body for control by the user.

FIG. 2 illustrates device 2 including elements which may be used with osteotomy device 1, by insertion therethrough; said device 2 may be optionally connected to device 1. Alternatively, device 2 may be permanently attached to device 1 by inserting distal end of device 2 through proximal hole 2000 of device 1 form a third device.

### DETAILED DESCRIPTION OF THE INVENTION

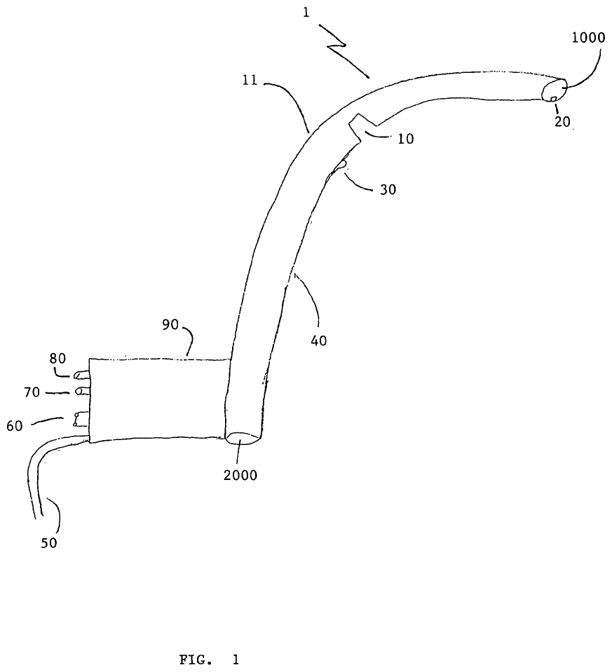

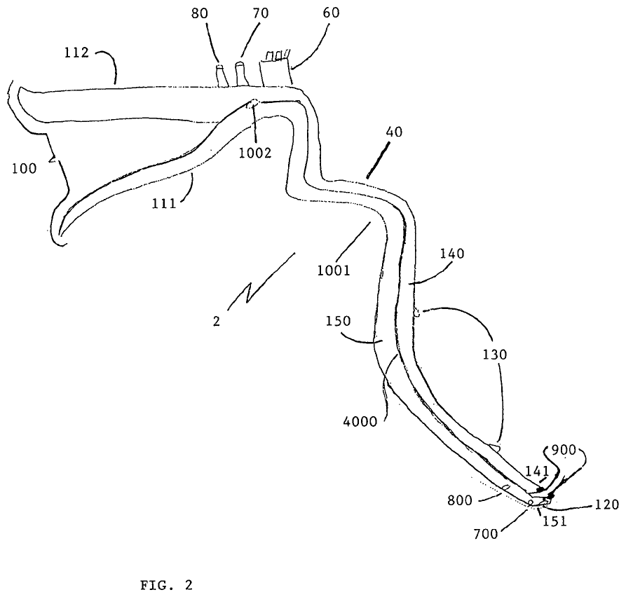

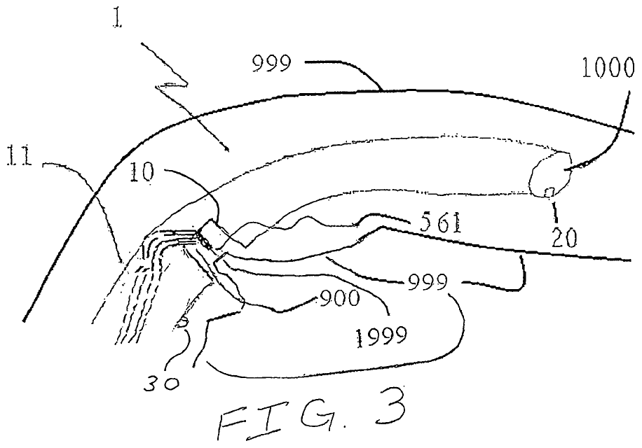

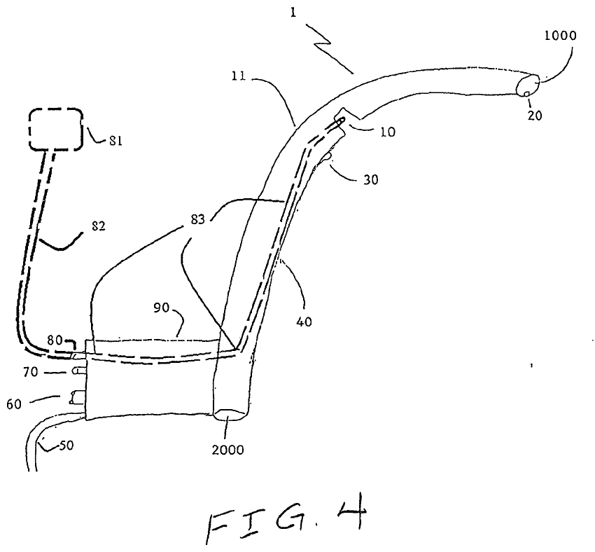

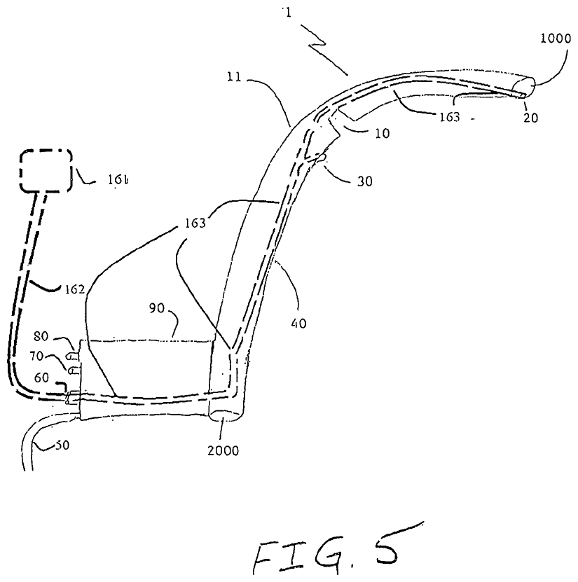

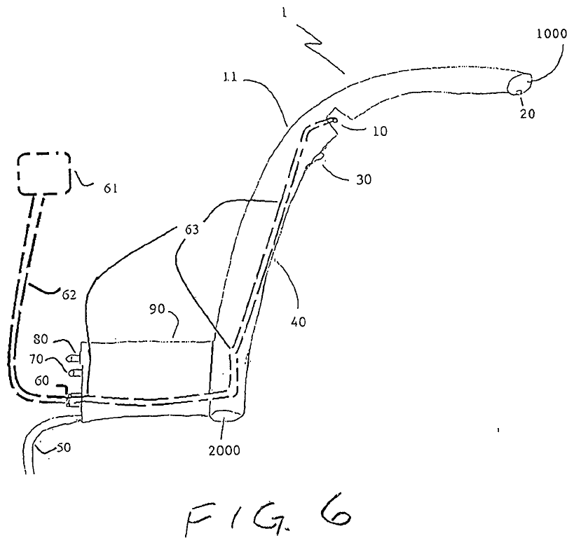

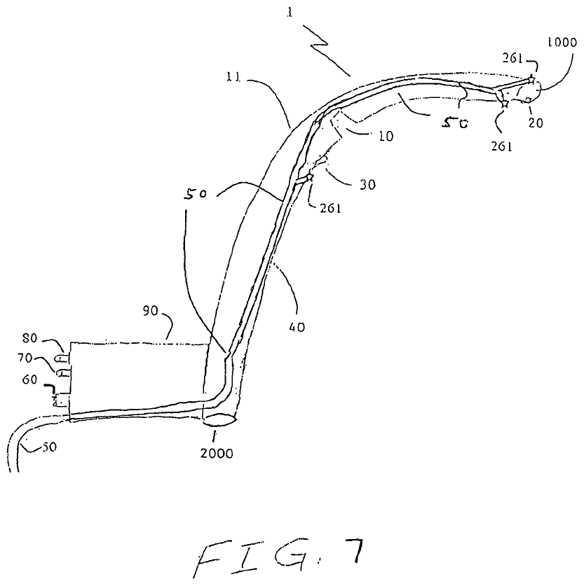

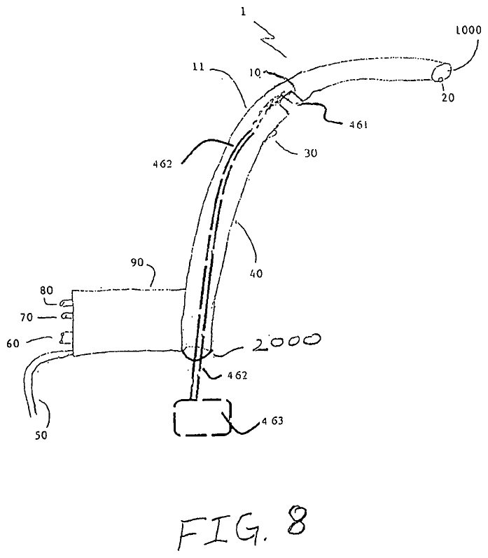

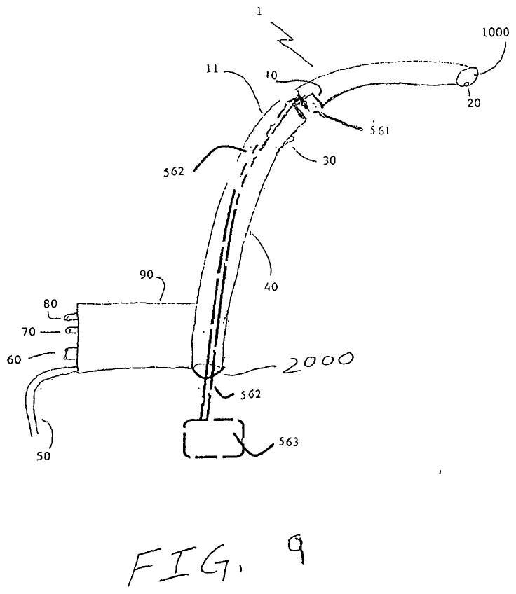

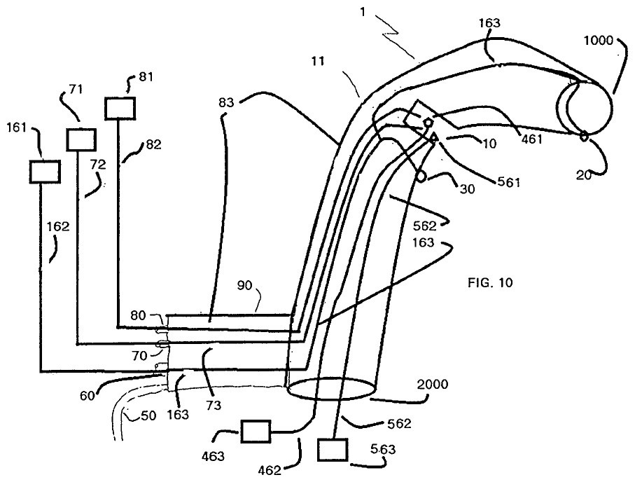

Now referring to FIG. 1, herein is described an osteotomy device 1 is composed of an elongate, straw-like cylinder 11 with proximal opening 2000 located outside the patient's body, and distal hole 1000 located inside the patient's body. Between proximal hole 2000 and distal hole 1000 is a side-slit 10 along the outside of cylinder element 11. Additionally, device 1 includes a handle 90, attached to cylinder 11. Handle 90 is optionally detachable when element 2 (illustrated in FIG. 2) is inserted through cylinder 11 via proximal hole 2000, and thereby rendering optional handle 90 redundant.

More particularly, cylinder 11 is composed of hard but flexible material suitable for protecting tissue proximal to a target surgical site. It is formed, optionally, of a biocompatible metal, plastic, or other suitable material, that is preferentially but optionally malleable. The cylinder including at least one side slit 10, wherein side slit 10 serves as a conduit to facilitate an osteotomy while the cylinder 11 helps to guide a cutting instrument in the proper orientation, and further serves to protect surrounding tissue from injury. Optional protective or hydrogel coating 40 assists insertion and further protects device and patient from irritation or injury.

Continuing with reference to FIG. 1, removable handle 90 includes optional cable for communicating with at least two cameras: end camera 20 and side camera 30 and optional lights (not shown); endoscopes (not shown) are delivered via proximal hole 2000. Additionally, handle 90 includes cable 50 for communicating and powering lights (not shown), cameras 20 and 30, and endoscopy scopes (not shown); optional cauterization-communication port 60, optional aspiration port 70, and optional irrigation port 80. Said handle 90 communicates with end camera 20 and side camera 30, as well as providing ports for aspiration via optional aspiration port 70 and irrigation via optional irrigation port 80. In a preferred embodiment, however, cable 50 is directed through handle 90; cauterization-communication port 60, aspiration port 70, and irrigation port 80 are all included.

Elongate cylinder 11 is disposed with at least two cameras/endoscope 20 and 30 along its length, with illumination. The camera or endoscope and lighting may be disposed at the distal tip and/or anywhere along the length of the exterior to facilitate direct, minimally invasive, and magnified visualization. The image may optionally appear in high-definition (HD) and/or three-dimensionally (3D). Out of side-hole 10 sonic, laser and other conventional cutting devices may be deployed simultaneously with irrigation and aspiration elements (not shown) but are introduced via optional ports 60, 70 and 80. More particularly, end camera 20 and side camera 30, as well as lights (not shown), endo-scope (not shown), and cutting devices (not shown) communicate through removable handle 90 through cable 50.

Now referring to FIG. 2, device 2 has a proximal end handle 100, and a distal end camera 120, proximal to distal camera 120 are optional cauterization elements 900, aspiration hole 700, irrigation hole 800, and at least one optional side camera 130. Said distal end is integrated with device 1 from FIG. 1 by introducing distal camera end of device 2 into proximal hole 2000, and pushing device 2 through cylinder 11 until distal camera end is proximal to side-slit 10. Between handle 10 and distal camera end of device 2 are paired elements 1001 which is composed of upper element 140 and lower element 150 which slide against each other in response to the closing of handle 100 around joint 1002. More particularly, extending out from handle 100 are paired elements 1001. Handle 100 further comprises a combination element 1001 of upper sliding element 140 and lower sliding element 150 which, when activated, allows sliding elements 140 and 150 to slide against each other along interface 4000. When bottom handle element 111, and top handle element 112 are pinched together, lower element 150 retracts relative to upper element 140 toward handle 100, and the distal end 151 of element 150 moves closer to distal end 141 of element 140. When bottom element 111 is proximal to top element 112, distal end 141 of element 140 is proximal to distal end 151 of lower element 150. Together, distal ends of elements 141 and 151 form a cutting or clipping device. When 141 and 151 are opened and closed, device 2 is capable of clipping tissue or cutting bone. Furthermore, optional cauterization-communication port 60 allows external cameral systems to communicate with side-camera 130 and end-camera 120.

The above-described osteotomy device 1 is adaptable to a number of embodiments, desirable or necessary for differing procedures.

In a preferred embodiment, the cylinder 11 is malleable, rendering cylinder 11 actively steerable. One or more of the optional embodiments of the osteotomic cylinder 11 may incorporate malleability and/or steerability.

In an optional embodiment, the cylinder 11 is incorporated with suction and/or irrigation functionality (not shown).

In another embodiment, the osteotomy cylinder 11 incorporates monopolar cautery and/or bipolar cautery (not shown). Said cautery capability may be disposed upon the tip or at the at least one slit of the cylinder.

In an optional embodiment, cylinder 11 incorporates a Kerrison bone punch. In this embodiment, the Kerrison punch may optionally be manual or powered.

In an optional embodiment, cylinder 11 incorporates a Kerrison rongeur (not shown). In this embodiment, the Kerrison rongeur may optionally be manual or powered.

In an optional embodiment, cylinder 11 incorporates a drill (not shown), optionally manual or powered. Cylinder 11 may be optimized to allow passage of a drill therethrough, for protected minimally invasive osteotomy.

In an optional embodiment, cylinder 11 incorporates an ultrasonic bone-cutter (not shown), optionally optimized to allow passage of a bone-cutter therethrough, for protected minimally invasive osteotomy.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. An osteotomy device capable of simultaneously connecting to three external systems comprising:
a handle having three ports and a cable;
an elongate cylinder providing tissue-protection, having an end camera, a side camera, a distal hole, a proximal bole, and a side-slit;
wherein said handle is affixed to said elongate cylinder in a substantially perpendicular orientation; and
wherein said osteotomy device is capable of introducing said external systems via both one of said ports or said proximal hole.

2. The osteotomy device of claim 1, wherein said cylinder is malleable.

3. The osteotomy device of claim 1, wherein said cylinder is steerable.

4. The osteotomy device of claim 1, wherein said handle is detachable from said elongate cylinder.

5. The osteotomy device of claim 1, wherein one said port is suitable for connecting with an external aspiration system.

6. The osteotomy device of claim 1, wherein one said port is suitable for connecting with an external irrigation system.

7. The osteotomy device of claim 1, wherein one said port is suitable for connecting with an external image-collection system.

8. The osteotomy device of claim 1, wherein one said port is suitable for connecting with an external cautery system.

9. (canceled)

10. The osteotomy device of claim 1, wherein said proximal hole is suitable for entry of an external Kerrison bone rongeur and said side slit is suitable for exit of said Kerrison bone rongeur,
wherein said elongate cylinder is capable of containing said Kerrison bone rongeur between said proximal hole and said side slit.

11. The osteotomy device of claim 1, wherein said proximal hole is suitable for entry of an external drill and side slit is suitable for exit of said drill,
wherein said elongate cylinder is capable of containing said drill between said proximal hole and said side slit.

12. The osteotomy device of claim 1, wherein said proximal hole is suitable for entry of an external ultrasonic bone cutter and said side slit is suitable for exit of said ultrasonic bone cutter,
wherein said elongate cylinder is capable of containing said ultrasonic bone cutter between said proximal hole and said side slit.

13. (canceled)

14. An osteotomy device capable of simultaneously attaching to three, comprising:
an elongate cylinder providing tissue-protection,
having an aspiration hole,
an irrigation hole,
a distal hole,
two side cameras disposed along said cylinder,
an upper clipping element,
a lower clipping element,
an upper sliding element,
a lower sliding element,
wherein said upper sliding element and said lower sliding element
communicate at a joint; and
wherein, said upper sliding element distally communicates with said upper clipping element, and said lower sliding element distally communicates at said lower clipping element to closeably effectuate a bone-cutting capability;
a handle, comprising five ports,
wherein one said five ports communicates with an aspiration system, one of said five ports communicates with an irrigation system, and
three of said five ports communicate with a cauterization-communication system; and
a bottom element and a top element, and said joint,
wherein said top element communicates with the proximal end of said upper sliding element, and said bottom element communicates with the proximal end of said lower sliding element.

15. The osteotomy device of claim 14, wherein said cylinder is malleable.

16. (canceled)

17. (canceled)

18. (canceled)

19. The osteotomy device of claim 14, further comprising a cautery disposed upon said elongate cylinder, wherein said cautery is selected from one of the following: a monopolar cautery, and a bipolar cautery,

20. (canceled)

21. The osteotomy device of claim 14, wherein said cylinder is steerable.

22. (canceled)

23. (canceled)

24. (canceled)

25. (canceled)

26. (canceled)

27. An osteotomy device capable of simultaneously connecting to at least three external systems, said device comprising:
a handle having three ports and a cable;
an elongate cylinder providing tissue-protection, having an end camera, at least
one side camera, a distal hole, a proximal hole, and at least one side-slit;
wherein said handle is affixed to said elongate cylinder.

28. The osteotomy device of claim 27, wherein said cylinder is steerable.

29. The osteotomy device of claim 27, wherein one said port is suitable for connecting with an external cautery system.

30. The osteotomy device of claim 27, wherein said cylinder is malleable.
